(19) United States
(12) Patent Application Publication
(10) Pub. No.: US 2026/0069295 A1
(43) Pub. Date: Mar. 12, 2026

(54) ASPIRATION THROMBECTOMY DEVICE

(71) Applicant: Boston Scientific Scimed, Inc., Maple Grove, MN (US)

(72) Inventors: Michael William Nagel, Andover, MN (US); Ryan Lee Reifsteck, New Hope, MN (US)

(73) Assignee: Boston Scientific Scimed, Inc., Maple Grove, MN (US)

(21) Appl. No.: 19/319,989

(22) Filed: Sep. 5, 2025

Related U.S. Application Data
(60) Provisional application No. 63/691,469, filed on Sep. 6, 2024.

(51) Int. Cl. A61B 17/22; A61M 25/00; A61M 25/01

(52) U.S. Cl. CPC A61B 17/22; A61M 25/0023; A61M 25/0136; A61B 2017/22038; A61B 2017/22079

(57) ABSTRACT

Example medical devices are disclosed herein. An example thrombectomy device includes a catheter assembly having an outer shaft having a proximal end region, a distal end region and a lumen, wherein the distal end region includes a first inflow orifice.

Further, the catheter assembly includes an inner shaft having a distal end region and a lumen extending within at least a portion of the lumen of the outer shaft, wherein the distal end region of the inner shaft includes a second inflow orifice. Further, the catheter assembly includes a guidewire shaft extending through the lumen of the inner shaft, a tip member coupled to a distal end of the guidewire shaft and a handle configured to shift the catheter assembly such that first inflow orifice, the second inflow orifice, or both the first inflow orifice and the second inflow orifice are in fluid communication with the lumen of the inner shaft.

### CROSS-REFERENCE TO RELATED APPLICATIONS

This application claims the benefit of priority under 35 U.S. C. § 119 of U.S. Provisional Application No. 63/691,469, filed Sep. 6, 2024, the entire disclosure of which is hereby incorporated by reference.

### TECHNICAL FIELD

The present disclosure pertains to medical devices, and methods for manufacturing medical devices. More particularly, the present disclosure pertains to thrombectomy devices.

### BACKGROUND

Thrombectomy is a procedure for removing thrombus from the vasculature of a patient. Mechanical and fluid-based systems can be used to remove thrombus. With fluid-based systems, an infusion fluid may be directed to a treatment area of a vessel with a catheter to dislodge the thrombus. In some instances, an effluent (e.g., the infusion fluid and/or blood) including the dislodged thrombus may be extracted from the vessel through the catheter. Of the known thrombectomy systems and methods, there is an ongoing need to provide alternative configurations, as well as methods of operating such thrombectomy systems.

### SUMMARY

This disclosure provides design, material, manufacturing method, and use alternatives for medical devices. An example thrombectomy device includes a catheter assembly. The catheter assembly includes an outer shaft having a proximal end region, a distal end region and a lumen, wherein the distal end region of the outer shaft includes a first inflow orifice. Further, the catheter assembly includes an inner shaft having a distal end region and a lumen, wherein the inner shaft extends within at least a portion of the lumen of the outer shaft, and wherein the distal end region of the inner shaft includes a second inflow orifice. Further, the catheter assembly includes a guidewire shaft extending through the lumen of the inner shaft, a tip member coupled to a distal end of the guidewire shaft and a handle configured to shift the catheter assembly such that first inflow orifice, the second inflow orifice, or both the first inflow orifice and the second inflow orifice are in fluid communication with the lumen of the inner shaft.

Alternatively or additionally to any of the examples above, wherein the first inflow orifice extends through a sidewall of the outer shaft.

Alternatively or additionally to any of the examples above, wherein the second inflow orifice extends through a sidewall of the inner shaft.

Alternatively or additionally to any of the examples above, wherein the distal end region of the outer shaft is spaced away from the tip member.

Alternatively or additionally to any of the examples above, wherein a distal end of the inner shaft abuts the tip member.

Alternatively or additionally to any of the examples above, wherein the handle includes a first actuation member configured to rotate the outer shaft relative to the inner shaft.

Alternatively or additionally to any of the examples above, wherein rotating the outer shaft radially aligns the first inflow orifice relative with the second inflow orifice.

Alternatively or additionally to any of the examples above, wherein the thrombectomy device is configured to aspirate thrombogenic material through both the first inflow orifice and the second inflow orifice.

Alternatively or additionally to any of the examples above, wherein the second inflow orifice includes a front-facing inflow orifice, and wherein the front-facing inflow orifice is in fluid communication with the lumen of the inner shaft.

Alternatively or additionally to any of the examples above, wherein the handle includes a first actuation member configured to translate the inner shaft relative to both the outer shaft and the tip member.

Alternatively or additionally to any of the examples above, wherein translating the inner shaft shifts the inner shaft between a first configuration in which the front-facing inflow orifice of the inner shaft engages the tip member and a second configuration in which the front-facing inflow orifice of the inner shaft is spaced away from the tip member.

Alternatively or additionally to any of the examples above, wherein translating the inner shaft from the first configuration to the second configuration closes the first inflow orifice.

Alternatively or additionally to any of the examples above, wherein the thrombectomy device is configured to aspirate thrombogenic material through the front-facing orifice.

Alternatively or additionally to any of the examples above, wherein the inner shaft includes a front-facing inflow orifice.

Alternatively or additionally to any of the examples above, wherein the handle includes a first actuation member configured to rotate the outer shaft relative to the inner shaft, and wherein the handle includes a second actuation member configured to translate the inner shaft relative to both the outer shaft and the tip member.

Alternatively or additionally to any of the examples above, wherein actuation of the first actuation member and the second actuation member configures the thrombectomy device to aspirate thrombogenic material through the first inflow orifice, the second inflow orifice and the front-facing orifice.

Another example thrombectomy system includes a pump coupled to a thrombectomy catheter assembly. The catheter assembly includes an outer shaft having a proximal end region, a distal end region and a lumen, wherein the distal end region of the outer shaft includes a first inflow orifice. Further, the catheter assembly includes an inner shaft having a distal end region and lumen, wherein the inner shaft extends within at least a portion of the lumen of the outer shaft, and wherein the distal end region of the inner shaft includes a second inflow orifice. Further, the catheter assembly includes a guidewire shaft extending through the lumen of the inner shaft, a tip member coupled to a distal end of the guidewire shaft and a handle configured to shift the catheter assembly such that first inflow orifice, the second inflow orifice, or both the first inflow orifice and the second inflow orifice are in fluid communication with the lumen of the inner shaft. Further, the pump is configured to apply a vacuum force within the lumen of the inner shaft.

Alternatively or additionally to any of the examples above, wherein the first inflow orifice extends through a sidewall of the outer shaft.

Alternatively or additionally to any of the examples above, wherein the second inflow orifice extends through a sidewall of the inner shaft.

An example method for aspirating a target tissue site within a body vessel includes advancing a thrombectomy device adjacent to the target tissue site, the thrombectomy device including an outer shaft having a proximal end region, a distal end region and a lumen, wherein the distal end region of the outer shaft includes a first inflow orifice. Further, the thrombectomy device includes an inner shaft having a distal end region and lumen, wherein the inner shaft extends within at least a portion of the lumen of the outer shaft, and wherein the distal end region of the inner shaft includes a second inflow orifice. Further, the thrombectomy device includes a guidewire shaft extending through the lumen of the inner shaft, a tip member coupled to a distal end of the guidewire shaft and a handle configured to shift the catheter assembly such that first inflow orifice, the second inflow orifice, or both the first inflow orifice and the second inflow orifice are in fluid communication with the lumen of the inner shaft. Additionally, the method includes applying a vacuum force to the inner lumen of the inner shaft.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a side view of an example thrombectomy device;

FIG. 2 is a longitudinal cross-sectional view of a distal end region of the thrombectomy device shown in FIG. 1;

FIG. 3 is a side view of a portion of the thrombectomy device shown in FIG. 1;

FIG. 4 illustrates a thrombectomy device positioned in a body lumen;

FIG. 5 is a longitudinal cross-sectional view of a distal end region of another example thrombectomy device;

FIG. 6 illustrates the thrombectomy device of FIG. 1 positioned in a body lumen;

FIG. 7 illustrates the thrombectomy device of FIG. 1 positioned in a body lumen;

FIG. 8 illustrates the thrombectomy device of FIG. 1 positioned in a body lumen;

FIG. 9 is a longitudinal cross-sectional view of a distal end region of another example thrombectomy device; and

FIG. 10 is a longitudinal cross-sectional view of a distal end region of another example thrombectomy device.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Thrombectomy devices and systems may be used to remove thrombus, plaques, lesions, clots, etc. from veins or arteries. Some thrombectomy devices may utilize specifically oriented high velocity saline jets to entrain fluid or clot material into and through a shaft of the catheter. Other thrombectomy systems may utilize one or more pressurized saline jets which travel backwards to create a low-pressure zone and a vacuum effect, whereby the vacuum pulls clot material into and through one or more apertures in the shaft of the device. In some instances, it may be beneficial to perform aspiration thrombectomy through a side hole in the shaft of a thrombectomy device. In other instances, it may be beneficial to perform aspiration thrombectomy through a front-facing inflow orifice of a thrombectomy device. In yet other instances, it may be beneficial to perform aspiration thrombectomy through both a side hole and a front-facing inflow orifice of a thrombectomy device. Thrombectomy systems that allow aspiration through a side hole, a front-facing inflow orifice or both a side hole and a front-facing inflow orifice of a thrombectomy device are disclosed herein.

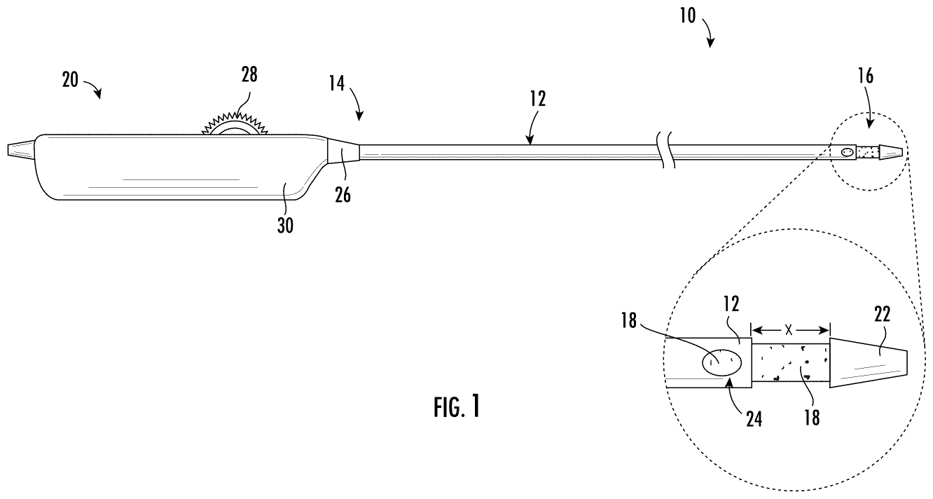

FIG. 1 is a side view of an example thrombectomy device 10. The thrombectomy device 10 may include an outer shaft 12 having a proximal end region 14 and a distal end region 16. A handle 20 may be coupled to the proximal end region 14 of the catheter shaft 12. The handle 20 may include a number of a features. For example, the handle 20 may include a thumbwheel 28 coupled to a handle housing 30. Additionally, the handle 20 may include a rotational actuation member 26 coupled to an outer surface of the outer shaft 12. As will be discussed in greater detail herein, the rotational actuation member 26 may be configured to rotate (and thereby rotate the outer shaft 12) relative to the handle housing 30. Further, it can be appreciated that the thumbwheel 28 may be configured to rotate relative to the handle housing 30.

Further, the detailed view of FIG. 1 illustrates the outer shaft 12 may include one or more orifices (e.g., apertures, openings, etc.) positioned along the distal end region 16 of the outer shaft 12. For example, the distal end region 16 of the outer shaft 12 may have a first inflow orifice 24. The first inflow orifice 24 may be disposed proximally of the distal end of the outer shaft 12.

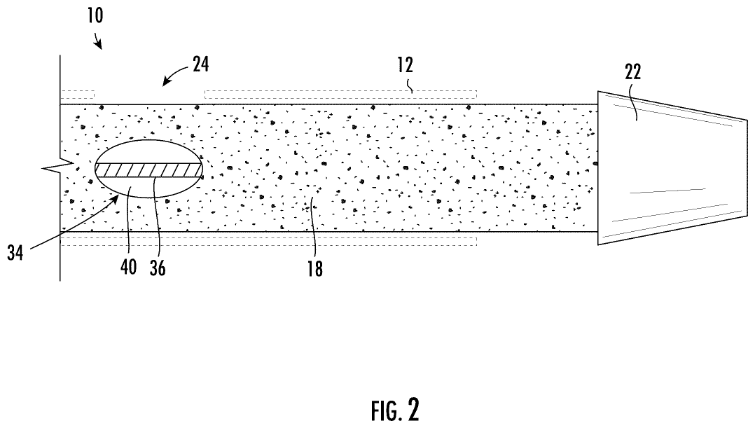

Further, FIG. 1 illustrates that the thrombectomy device 10 may include an inner catheter 18 extending with a lumen of the outer shaft 12. Additionally, FIG. 1 illustrates that the distal end region 16 of the thrombectomy device 10 may include a tip member 22. As will be discussed in greater detail herein, the tip member 22 may be attached to a distal end of a guidewire shaft 36 (shown in FIG. 2) which extends through a lumen of the inner catheter 18. FIG. 1 further illustrates that the distal end of the outer shaft may be spaced away from the tip member a distance “X.” In some examples, the distance X may be about 0 mm to about 26 mm, or about 4 mm to about 22 mm, or about 8 mm to about 18 mm, or about 10 mm to about 16 mm.

FIG. 2 is a longitudinal cross-sectional view of a distal end region of the thrombectomy device 10 shown in FIG. 1. It can be appreciated that the view shown in FIG. 2 illustrates the distal end region of the thrombectomy device 10 shown in FIG. 1 rotated 45 degrees into the page.

FIG. 2 further illustrates the inner shaft 18 extending within a lumen 40 of the outer shaft 12. Further, FIG. 2 illustrates that the distal end region of the inner shaft 18 may include one or more second inflow orifices 34 (e.g., apertures, openings, etc.) positioned along the distal end region of the inner shaft 18. For example, the distal end region of the inner shaft 12 may include a second inflow orifice 34. FIG. 2 further illustrates the guidewire shaft 36 extending within the lumen 40 of the inner shaft 18. As discussed herein, a distal end of the guidewire shaft 36 may be attached to the tip member 22. FIG. 2 illustrates the distal end of the outer shaft 12 spaced away from tip member 22.

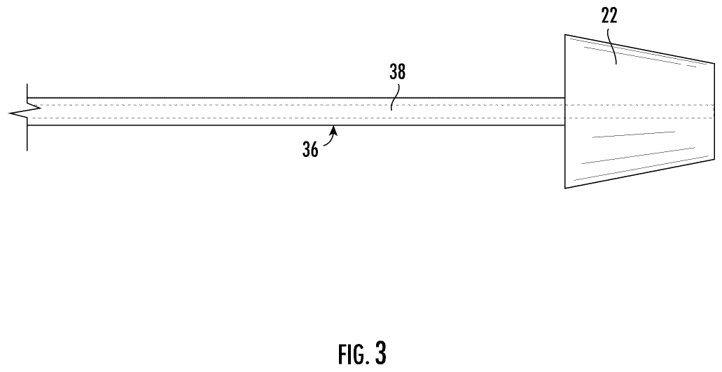

FIG. 3 illustrates the guidewire shaft 36 attached to the tip member 22. It can be appreciated from FIG. 3 that the guidewire shaft 36 may include a guidewire lumen 38. In some examples, the guidewire lumen 38 extends through the tip 22, thereby permitting a guidewire to extend through both the guidewire lumen 38 and the tip 22. Accordingly, this configuration may permit the thrombectomy device 10 to be tracked over a guidewire to a target treatment site. In other examples, the guidewire shaft 36 and the tip 22 may be solid, whereby the thrombectomy device 10 would be utilized without a guidewire.

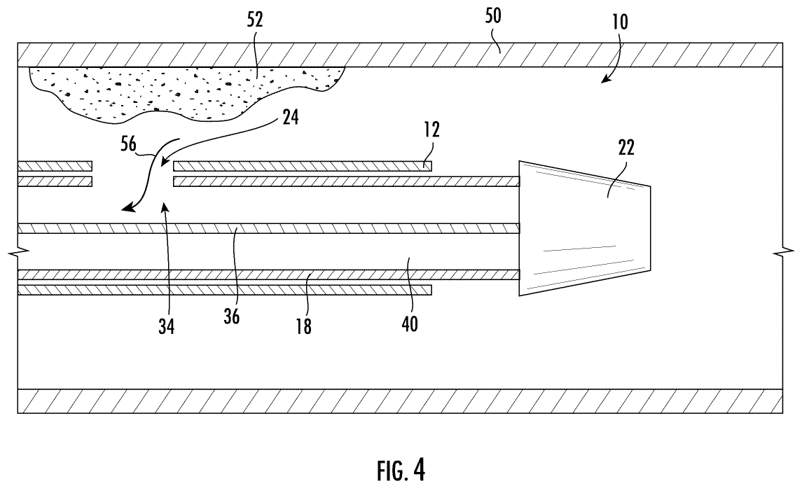

FIGS. 4-7 are cross-sectional views that schematically depict how the thrombectomy device 10 may be used to aspirate material (e.g., embolic material, thrombus/thrombogenic material, stenotic material, etc.) from a patient.

FIG. 4 illustrates a portion of the thrombectomy device 10 positioned in a body vessel 50 of a patient. Further, FIG. 4 illustrates that the thrombectomy device 10 has been positioned in the body vessel 50 of a patient such that the first inflow orifice 24 of the outer shaft 12 has been positioned adjacent to thrombus 52 located within the body vessel 50. Additionally, it can be appreciated from FIG. 4 that the first inflow orifice 24 of the outer shaft 12 has been rotationally aligned with the second inflow orifice 34 of the inner member 18. Further, it can be appreciated that, when in the configuration illustrated in FIG. 4, the tip member 22 may be positioned against the distal end of the inner shaft 18, thereby sealing the lumen of the inner shaft 18 and preventing the flow of material (e.g., blood, thrombogenic material, etc.) into the lumen of the inner shaft 18.

In some examples, the first inflow orifice 24 of the outer shaft 12 may be aligned with the second inflow orifice 34 of the inner shaft 18 via actuation of the rotational actuation member 26. For example, the distal end region of the thrombectomy device 10 may be tracked to the target thrombus site 52 in the configuration shown in FIG. 2, whereby the first inflow orifice 24 of the outer shaft 12 is radially offset (e.g., rotationally offset, circumferentially offset, etc.) from the second inflow orifice 34 of the inner shaft 18. After being tracked to a position adjacent the thrombus 52 shown in FIG. 4, a user may grip the rotational actuation member 26 on the handle 20 and rotate the outer shaft 12 to a configuration in which the first inflow orifice 24 of the outer shaft 12 has been radially aligned with the second inflow orifice 34 of the inner member 18 (as shown in FIG. 4). Further, it can be appreciated from FIG. 4 that once the first inflow orifice 24 of the outer shaft 12 has been radially aligned with the second inflow orifice 34 of the inner member 18, a vacuum force may be applied to the lumen 40 of the inner shaft 18, thereby pulling thrombogenic material 52 through both of the first and second inflow orifices 24, 34 and into the lumen 40 of the inner shaft 18, whereby it may be aspirated out of the patient. The flow of the aspirated thrombotic material 52 through both of the first and second inflow orifices 24, 34 and into the lumen 40 of the inner shaft 18 and out of the patient is shown by the reference arrow 56 in FIG. 4. As discussed herein, FIG. 4 illustrates that positioning the tip member 22 against the distal end of the inner shaft 18 may prevent the aspiration of thrombotic material 52 through the lumen of the inner shaft 18.

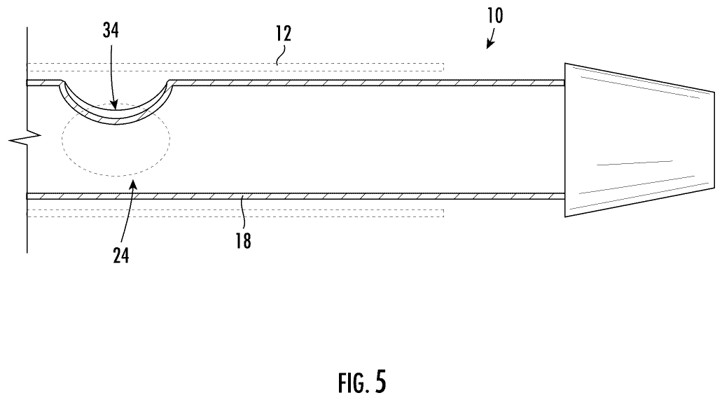

FIG. 5 illustrates that for examples in which the outer shaft 12 may be rotated relative to the inner shaft 18, the first inflow orifice 24 of the outer shaft 12 may overlap to any extent, degree, etc. relative to the second inflow orifice 34 of the inner shaft 18. For example, FIG. 5 illustrates the outer shaft 12 having been rotated relative to the inner shaft 18 to an extent in which the first inflow orifice 24 of the outer shaft 12 partially overlaps the second inflow orifice 34 of the inner shaft 18. In comparison, the example illustrated in FIG. 2 shows the first inflow orifice 24 of the outer shaft 12 having no overlap with the second inflow orifice 34 of the inner shaft 18. Further, the example illustrated in FIG. 4 shows the first inflow orifice 24 of the outer shaft 12 completely aligned (e.g., complete overlap) with the second inflow orifice 34 of the inner shaft 18.

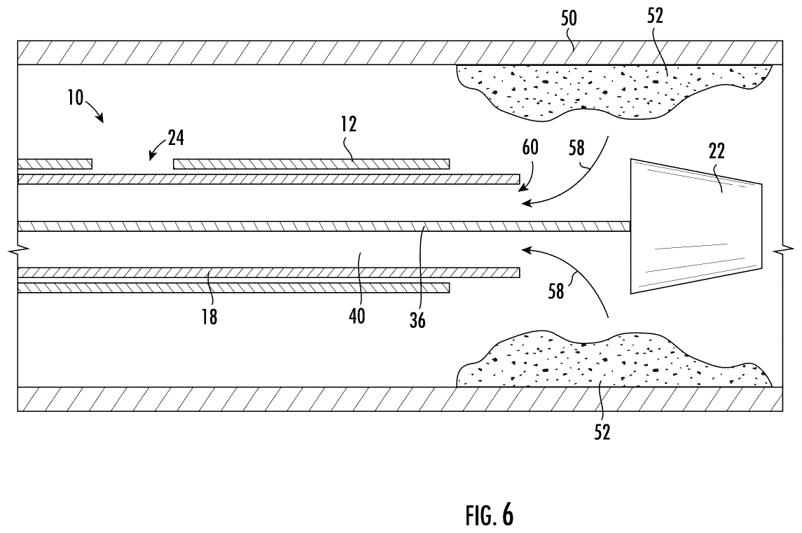

FIG. 6 illustrates another example cross-sectional view that schematically depicts how the thrombectomy device 10 may be used to aspirate material (e.g., embolic material, thrombus/thrombogenic material, stenotic material, etc.) from a patient. FIG. 6 illustrates a portion of the thrombectomy device 10 positioned in a body vessel 50 of a patient.

FIG. 6 illustrates that the thrombectomy device 10 has been positioned in the body vessel 50 of a patient such that the distal end of the outer shaft 12 and the distal end of the inner shaft 18 have been positioned adjacent to a thrombus 52 located within the body vessel 50. Additionally, it can be appreciated from FIG. 6 that the thrombectomy device has been positioned adjacent the target thrombus 52 in a configuration in which the first inflow orifice 24 of the outer shaft 12 is radially offset (e.g., rotationally offset, circumferentially offset, etc.) from the second orifice 34 of the inner shaft 18 (such as the configuration shown in FIG. 2). Accordingly, it can be appreciated that, when in the configuration illustrated in FIG. 6, the outer surface of the inner shaft 18 may prevent flow of material (e.g., blood, thrombogenic material, etc.) through the first inflow orifice 24 of the outer shaft 12. For example, if a vacuum force is applied to lumen 40 of the inner shaft 18, the offset (e.g., rotationally offset, circumferentially offset, etc.) of the first inflow orifice 24 from the second inflow orifice 34 of the inner shaft 18 may prevent flow of material (e.g., blood, thrombogenic material, etc.) through the first inflow orifice 24 and into the lumen 40 of the inner shaft 18.

Additionally, FIG. 6 further illustrates that, in some examples, the guidewire 36 shaft may be actuated in a proximal-to-distal direction, thereby spacing the tip member 22 away from the distal end of the outer shaft 12 and the distal end of the inner shaft 18. For example, the thumbwheel 28 of the handle 20 may be coupled to the guidewire shaft 36 whereby actuation (e.g., clockwise rotation) of the thumbwheel 28 may advance the guidewire shaft 36 in a proximal-to-distal direction, thereby advancing the tip member 22 distally relative to the distal end of the outer shaft 12 and the distal end of the inner shaft 18. It can be further appreciated that the thumbwheel 28 may be configured such that reversing rotation of the thumbwheel (e.g., counterclockwise rotation) may retract the guidewire shaft 36 in a distal-to-proximal direction, thereby retracting the tip member 22 to a position in which it abuts the distal end of the inner shaft 18 (such as the configuration shown in FIG. 2).

Further, it can be appreciated from FIG. 6 that once the tip member 22 has been advanced distally relative to the distal end of the inner shaft 18, a vacuum force may be applied to the lumen 40 of the inner shaft 18, thereby pulling thrombotic material through a front-facing inflow orifice 60 of the inner shaft 18 and into the lumen 40 of the inner shaft 18 whereby it may be aspirated out of the patient. The flow of the aspirated thrombotic material through the front-facing inflow orifice 60 and into the lumen 40 of the inner shaft 18 and out of the patient is shown by the reference arrows 58 in FIG. 6. As set forth herein, the term “front-facing” may include, but is not limited to, a generally distal-facing opening, an angled distal-facing opening, a slightly-angled distal facing opening, a tapered distal-facing opening, a forward-facing opening, etc.

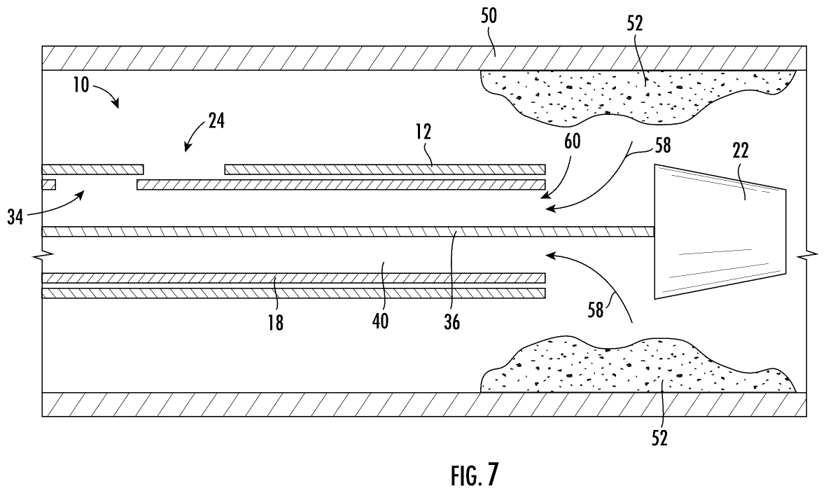

FIG. 7 illustrates another example cross-sectional view that schematically depicts how the thrombectomy device 10 may be used to aspirate material (e.g., embolic material, thrombus/thrombogenic material, stenotic material, etc.) from a patient. FIG. 7 illustrates a portion of the thrombectomy device 10 positioned in a body vessel 50 of a patient.

FIG. 7 illustrates that the thrombectomy device 10 has been positioned in the body vessel 50 of a patient such that the distal end of the outer shaft 12 and the distal end of the inner shaft 18 have been positioned adjacent to a thrombus 52 located within the body vessel 50. Additionally, FIG. 7 illustrates that the inner shaft 18 may be actuated in a distal-to-proximal direction, thereby retracting the end of the inner shaft 18 away from the tip member 22. For example, the thumbwheel 28 of the handle 20 may be coupled to the inner shaft 18 whereby actuation (e.g., counter-clockwise rotation) of the thumbwheel 28 may retract the inner shaft 18 in a distal-to-proximal direction, thereby retracting the inner shaft 18 proximally relative to the tip member 22. It can be appreciated from FIG. 7 that once the inner shaft 18 has been retracted proximally relative to the outer shaft 12 and the tip member 22, the first inflow orifice 24 of the outer shaft 12 may be longitudinally offset from the second inflow orifice 34 of the inner shaft 18. Accordingly, it can be appreciated from FIG. 7 that when the inner shaft 18 is in a retracted configuration, no matter how the second inflow orifice 34 is radially aligned with the first inflow orifice 24, the longitudinal offset of the first inflow orifice 24 relative to the second inflow orifice 34 of the inner shaft 18 may always prevent flow of material (e.g., blood, thrombogenic material, etc.) through the first inflow orifice 24 and into the lumen 40 of the inner shaft 18.

It can be further appreciated that the thumbwheel 28 may be configured such that reversing rotation of the thumbwheel (e.g., clockwise rotation) may advance the inner shaft 18 in a proximal-to-distal direction, thereby advancing the distal end of the inner shaft 18 to a position in which it abuts the tip member 22 (such as the configuration shown in FIG. 2).

Additionally, it can be appreciated from FIG. 7 that once the inner shaft 18 has been retracted proximally relative to the outer shaft 12 and the tip member 22 (thereby longitudinally offsetting the first inflow orifice 24 of the outer shaft 12 from the second inflow orifice 34 of the inner shaft 18), a vacuum force may be applied to the lumen 40 of the inner shaft 18. The vacuum force may pull thrombotic material through the front-facing inflow orifice 60 of the inner shaft 18 and into the lumen 40 of the inner shaft 18, whereby it may be aspirated out of the patient. The flow of the aspirated thrombotic material through the front-facing inflow orifice 60 and into the lumen 40 of the inner shaft 18 and out of the patient is shown by the reference arrows 58 in FIG. 7.

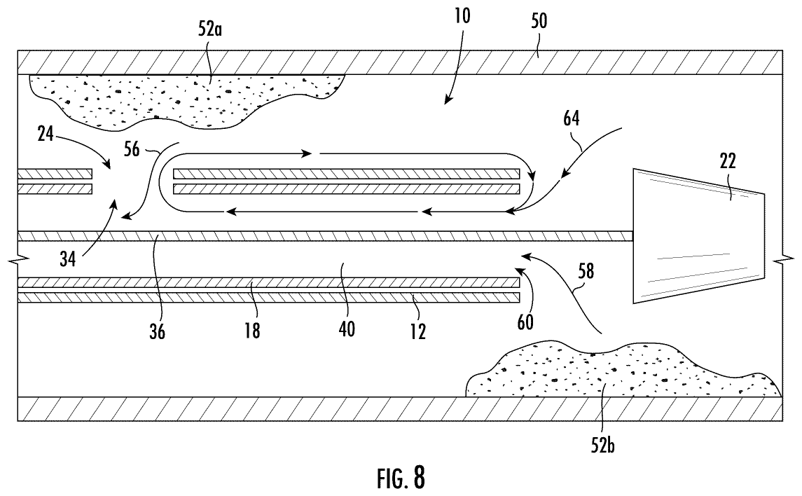

FIG. 8 illustrates another example cross-sectional view that schematically depicts how the thrombectomy device 10 may be used to aspirate material (e.g., embolic material, thrombus/thrombogenic material, stenotic material, etc.) from a patient. FIG. 8 illustrates a portion of the thrombectomy device 10 positioned in a body vessel 50 of a patient.

Like the example discussed herein with respect to FIG. 4, FIG. 8 illustrates that the thrombectomy device 10 has been positioned in the body vessel 50 of a patient such that the first inflow orifice 24 of the outer shaft 12 has been positioned adjacent to thrombus 52a located within the body vessel 50.

FIG. 8 illustrates an example configuration in which the first inflow orifice 24 may be positioned along the inner shaft 18 such that retraction of the inner shaft 18 may space the front-facing orifice 60 away from the tip member 22 while also aligning the first inflow orifice 34 with the second inflow orifice 24. For example, FIG. 8 illustrates a configuration in which the rotation of the outer shaft 12 combined with retraction of the inner shaft 18, may radially and longitudinally align the first inflow orifice 34 with the second inflow orifice 24, while also exposing the front-facing orifice 60. For example, FIG. 8 illustrates that after retraction of the inner shaft 18, the first inflow orifice 24 has been radially and longitudinally aligned with the second inflow orifice 34. It can be appreciated from FIG. 8 that once the first inflow orifice 24 of the outer shaft 12 has been longitudinally and radially aligned with the second inflow orifice 34 of the inner member 18, a vacuum force applied to the lumen 40 of the inner shaft 18 may pull thrombogenic material 52a through both of the first and second inflow orifices 24, 34 and into the lumen 40 of the inner shaft 18, whereby it may be aspirated out of the patient. The flow of the aspirated thrombotic material 52a through both of the first and second inflow orifices 24, 34 and into the lumen 40 of the inner shaft 18 and out of the patient is shown by the reference arrow 56 in FIG. 8.

Additionally, like the example discussed herein with respect to FIGS. 6-7, FIG. 8 illustrates that along with the alignment of the first inflow orifice 24 with the second inflow orifice 34, the retraction of the inner shaft 18 in a distal-to-proximal direction may space the end of the inner shaft 18 away from the tip member 22, thereby exposing the front-facing orifice 60 to the inner lumen 40. For example, actuation (e.g., counter-clockwise rotation) of the thumbwheel 28 may retract the inner shaft 18 in a distal-to-proximal direction, thereby retracting the inner shaft 18 relative to the tip member 22. It can be appreciated that in the example show in FIG. 8, retraction of the inner shaft 18 (along with rotation of the outer shaft via the rotational actuation member 26) may simultaneously longitudinally align the first inflow orifice 24 and the second inflow orifice 34, while also retracting the end of the inner shaft 18 away from the tip member 22, thereby exposing the front-facing orifice 60 to the inner lumen 40.

It can be appreciated from FIG. 8 that once the inner shaft 18 has been retracted proximally relative to the tip member 22, a vacuum force applied to the lumen 40 of the inner shaft 18 may also pull thrombotic material 52b through the front-facing inflow orifice 60 of the inner shaft 18 and into the lumen 40 of the inner shaft 18 whereby it may be aspirated out of the patient. The flow of the aspirated thrombotic material 52b through the front-facing inflow orifice 60 and into the lumen 40 of the inner shaft 18 and out of the patient is shown by the reference arrow 58 in FIG. 8.

FIG. 8 further illustrates that aspiration of thrombogenic material 52a through the first and second inflow orifices 24, 34 may occur simultaneously with the aspiration of thrombogenic material 52b through the front-facing inflow orifice 60 of the inner shaft 18. Additionally, FIG. 8 illustrates that this simultaneous aspiration of fluid (including thrombogenic material) into the lumen 40 may generate an aspiration force that can draw entrainment material into the lumen 40 through the front-facing inflow orifice 60. The material drawn into the lumen 40 may be aspirated through the lumen 40 and out of a patient. In addition or in the alternative, some or all of the thrombogenic material 52b drawn into the lumen 40 may exit the outer shaft 12 through the first and second orifices 24, 34. This material may recirculate and the action of recirculation may help to break up the thrombogenic material 52a in order to ease removal. For example, the material may enter the front-facing inflow orifice 60 where it can be aspirated from the patient and/or further recirculated.

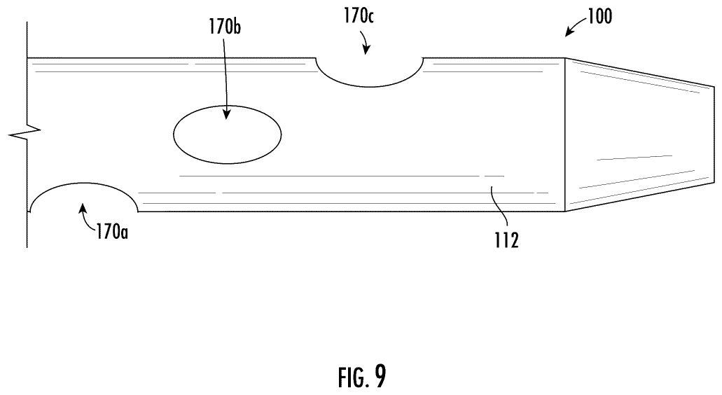

FIG. 9 illustrates another example thrombectomy device 100 that may include an outer shaft 112. The example outer shaft 112 may be similar in form and function to the outer shaft 12 described herein. Additionally, FIG. 9 illustrates that the outer shaft 112 may include a plurality of first inflow orifices 170a, 170b, 170c extending along the longitudinal axis of the shaft 112. FIG. 9 illustrates that each of the first inflow orifices 170a, 170b, 170c may be spaced away along the longitudinal axis of the shaft 112. For example, the first inflow orifice 170b may be distal relative to the proximal-most first inflow orifice 170a, and the first inflow orifice 170b may be proximal relative to the first inflow orifice 170c. Further, FIG. 9 illustrates that each of the first inflow orifices 170a, 170b, 170c may be radially offset from one another. For example, each of the first inflow orifices 170a, 170b, 170c may be radially offset approximately 45 degrees relative to one another. While FIG. 9 illustrates that the outer shaft including three first inflow orifices 170a, 170b, 170c, this is not intended to be limiting. Rather, the outer shaft 112 may include 1, 2, 3, 4, 5, 6, 7, 8 or more first inflow orifices which are longitudinally and radially offset from one another in any arrangement.

Additionally, for clarity purposes an inner shaft is not shown positioned in the lumen of the outer shaft 112 shown in FIG. 9. However, it can be appreciated that an inner shaft may be configured to be positioned within the lumen of the shaft 112, whereby the inner shaft may include any arrangement of second inflow orifices that are aligned and/or overlap with the first inflow orifices 170a, 170b, 170c of the outer shaft 112 in configurations similar to that described herein with respect to FIGS. 4-8. The outer shaft 112 may be rotated to align the first inflow orifices 170a, 170b, 170c relative to the second orifices of an inner shaft positioned with the lumen of the outer shaft 112 in a manner similar to that described herein with respect to FIGS. 4-8.

It can be appreciated that the size of the first inflow orifices 170a, 170b, 170c of the outer shaft 112 may be different than the size of second orifices of an inner shaft positioned with the lumen of the outer shaft 112. For example, the size of the first inflow orifices 170a, 170b, 170c of the outer shaft 112 may be larger than the size of second inflow orifices of an inner shaft positioned with the lumen of the outer shaft 112. Additionally, the size of the first inflow orifices 170a, 170b, 170c of the outer shaft 112 may be smaller than the size of second inflow orifices of an inner shaft positioned with the lumen of the outer shaft 112.

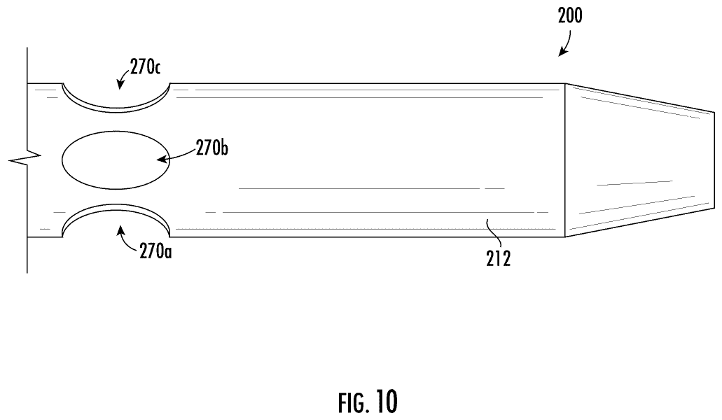

FIG. 10 illustrates another example thrombectomy device 100 that may include an outer shaft 212. The example outer shaft 212 may be similar in form and function to the outer shafts 12, 112 described herein. Additionally, FIG. 10 illustrates that the outer shaft 212 may include a plurality of first inflow orifices 270a, 270b, 270c extending along the longitudinal axis of the shaft 212. FIG. 10 illustrates that each of the first inflow orifices 270a, 270b, 270c may be longitudinally aligned with one another along the longitudinal axis of the shaft 212. Further, FIG. 10 illustrates that each of the first inflow orifices 270a, 270b, 270c may be radially offset from one another. For example, each of the first inflow orifices 270a, 270b, 270c may be radially offset approximately 45 degrees relative to one another. While FIG. 10 illustrates that the outer shaft including three first inflow orifices 270a, 270b, 270c, this is not intended to be limiting. Rather, the outer shaft 212 may include 1, 2, 3, 4, 5, 6, 7, 8 or more first inflow orifices which are longitudinally aligned and radially offset from one another.

Additionally, for clarity purposes an inner shaft is not shown positioned in the lumen of the outer shaft 212 shown in FIG. 10. However, it can be appreciated that an inner shaft may be configured to be positioned within the lumen of the shaft 212, whereby the inner shaft may include any arrangement of second inflow orifices that are aligned and/or overlap with the first inflow orifices 270a, 270b, 270c of the outer shaft 212 in configurations similar to that described herein with respect to FIGS. 4-8. The outer shaft 212 may be rotated to align the first inflow orifices 270a, 270b, 270c relative to the second orifices of an inner shaft positioned with the lumen of the outer shaft 212 in a manner similar to that described herein with respect to FIGS. 4-8.

It can be appreciated that the size of the first inflow orifices 270a, 270b, 270c of the outer shaft 212 may be different than the size of second orifices of an inner shaft positioned with the lumen of the outer shaft 212. For example, the size of the first inflow orifices 270a, 270b, 270c of the outer shaft 212 may be larger than the size of second orifices of an inner shaft positioned with the lumen of the outer shaft 212. Additionally, the size of the first inflow orifices 270a, 270b, 270c of the outer shaft 212 may be smaller than the size of second orifices of an inner shaft positioned with the lumen of the outer shaft 212.

It can be appreciated that in any of the examples described herein, one or more radiopaque markers (or other types of markers including, but not limited to, a line, a notch, etc.) may be positioned (e.g., aligned) on one or more components of the thrombectomy devices 10, 100, 200 to align any first inflow orifice on one component (e.g., the outer shaft 12) with a second inflow orifice on another component (e.g., the inner shaft 18). It can be appreciated that as the outer shaft 12 is rotated, the alignment of the two or more radiopaque markers may convey to a user that the first inflow orifice (e.g., orifice 24) on one component (e.g., the outer shaft 12) may be aligned with a second inflow orifice (e.g., orifice 34) on another component (e.g., the inner shaft 18).

Additionally, in some examples, the handle 20 may include one of more indicators which provide information regarding the alignment of any first inflow orifice on one component (e.g., the outer shaft 12) with a second inflow orifice on another component (e.g., the inner shaft 18). It can be appreciated that as the outer shaft 12 is rotated, the alignment of the two or more indicators on the handle 20 may convey to a user that the first inflow orifice (e.g., orifice 24) on one component (e.g., the outer shaft 12) may be aligned with a second inflow orifice (e.g., orifice 34) on another component (e.g., the inner shaft 18).

Further, while the above discussion disclosed embodiments directed toward an aspirational thrombectomy device, it can be appreciated that the thrombectomy devices disclosed herein and/or other devices disclosed herein may be utilized in other applications. For example, any of the devices disclosed herein may be utilized as a lytic delivery device.

The materials that can be used for the various components of the device 10, 100, 200 may include those commonly associated with medical devices. For simplicity purposes, the following discussion refers to the various components of the device 10, 100, 200. However, this is not intended to limit the devices and methods described herein, as the discussion may be applied to any components or devices disclosed herein.

The various components of the device 10, 100, 200 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the various components of the device 10, 100, 200 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the various components of the device 10, 100, 200 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the various components of the device 10, 100, 200 to achieve the same result.

In some embodiments, a degree of Magnetic Resonance Imaging (MRI) compatibility is imparted into the various components of the device 10, 100, 200. For example, the various components of the device 10, 100, 200, or portions thereof, may be made of a material that does not substantially distort the image and create substantial artifacts (e.g., gaps in the image). Certain ferromagnetic materials, for example, may not be suitable because they may create artifacts in an MRI image. The various components of the device 10, 100, 200, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed. It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A thrombectomy device, comprising:
a catheter assembly, including:
an outer shaft having a proximal end region, a distal end region and a lumen, wherein the distal end region of the outer shaft includes a first inflow orifice;
an inner shaft having a distal end region and lumen, wherein the inner shaft extends within at least a portion of the lumen of the outer shaft, and wherein the distal end region of the inner shaft includes a second inflow orifice;
a guidewire shaft extending through the lumen of the inner shaft;
a tip member coupled to a distal end of the guidewire shaft; and
a handle configured to shift the catheter assembly such that first inflow orifice, the second inflow orifice, or both the first inflow orifice and the second inflow orifice are in fluid communication with the lumen of the inner shaft.

2. The thrombectomy device of claim 1, wherein the first inflow orifice extends through a sidewall of the outer shaft.

3. The thrombectomy device of claim 2, wherein the second inflow orifice extends through a sidewall of the inner shaft.

4. The thrombectomy device of claim 3, wherein the distal end region of the outer shaft is spaced away from the tip member.

5. The thrombectomy device of claim 4, wherein a distal end of the inner shaft abuts the tip member.

6. The thrombectomy device of claim 5, wherein the handle includes a first actuation member configured to rotate the outer shaft relative to the inner shaft.

7. The thrombectomy device of claim 6, wherein rotating the outer shaft radially aligns the first inflow orifice relative with the second inflow orifice.

8. The thrombectomy device of claim 7, wherein the thrombectomy device is configured to aspirate thrombogenic material through both the first inflow orifice and the second inflow orifice.

9. The thrombectomy device of claim 2, wherein the second inflow orifice includes a front-facing inflow orifice, and wherein the front-facing inflow orifice is in fluid communication with the lumen of the inner shaft.

10. The thrombectomy device of claim 9, wherein the handle includes a first actuation member configured to translate the inner shaft relative to both the outer shaft and the tip member.

11. The thrombectomy device of claim 10, wherein translating the inner shaft shifts the inner shaft between a first configuration in which the front-facing inflow orifice of the inner shaft engages the tip member and a second configuration in which the front-facing inflow orifice of the inner shaft is spaced away from the tip member.

12. The thrombectomy device of claim 11, wherein translating the inner shaft from the first configuration to the second configuration closes the first inflow orifice.

13. The thrombectomy device of claim 12, wherein the thrombectomy device is configured to aspirate thrombogenic material through the front-facing orifice.

14. The thrombectomy device of claim 3, wherein the inner shaft includes a front-facing inflow orifice.

15. The thrombectomy catheter of claim 14, wherein the handle includes a first actuation member configured to rotate the outer shaft relative to the inner shaft, and wherein the handle includes a second actuation member configured to translate the inner shaft relative to both the outer shaft and the tip member.

16. The thrombectomy device of claim 15, wherein actuation of the first actuation member and the second actuation member configures the thrombectomy device to aspirate thrombogenic material through the first inflow orifice, the second inflow orifice and the front-facing orifice.

17. A thrombectomy system, comprising:
a pump coupled to a thrombectomy catheter assembly, wherein the thrombectomy catheter assembly includes:
an outer shaft having a proximal end region, a distal end region and a lumen, wherein the distal end region of the outer shaft includes a first inflow orifice;
an inner shaft having a distal end region and lumen, wherein the inner shaft extends within at least a portion of the lumen of the outer shaft, and wherein the distal end region of the inner shaft includes a second inflow orifice;
a guidewire shaft extending through the lumen of the inner shaft;
a tip member coupled to a distal end of the guidewire shaft; and
a handle configured to shift the catheter assembly such that first inflow orifice, the second inflow orifice, or both the first inflow orifice and the second inflow orifice are in fluid communication with the lumen of the inner shaft;
wherein the pump is configured to apply a vacuum force within the lumen of the inner shaft.

18. The thrombectomy device of claim 17, wherein the first inflow orifice extends through a sidewall of the outer shaft.

19. The thrombectomy device of claim 18, wherein the second inflow orifice extends through a sidewall of the inner shaft.

20. A method for aspirating a target tissue site within a body vessel, the method comprising:
advancing a thrombectomy device adjacent to the target tissue site, the thrombectomy device including:
an outer shaft having a proximal end region, a distal end region and a lumen, wherein the distal end region of the outer shaft includes a first inflow orifice;
an inner shaft having a distal end region and lumen, wherein the inner shaft extends within at least a portion of the lumen of the outer shaft, and wherein the distal end region of the inner shaft includes a second inflow orifice;
a guidewire shaft extending through the lumen of the inner shaft;
a tip member coupled to a distal end of the guidewire shaft; and
a handle configured to shift the catheter assembly such that first inflow orifice, the second inflow orifice, or both the first inflow orifice and the second inflow orifice are in fluid communication with the lumen of the inner shaft; and
applying a vacuum force to the inner lumen of the inner shaft.
